SEQUENCE LISTING (2 sequences)

SEQ ID NO: 1 - DNA, 2523 bp, Homo sapiens
gacaacttgagctgaaagcgaaaaactatgctgaccagattagcagacttgaagaaagagaagcagaactgaagaaggaatataatgcattacatcaaagacacactgagatgatccataattatatggaacatttagaaagaacaaaacttcatcagctctcagggagtgatcaactagaatccacagctcatagtagaattagaaaagaacgccctatatcattaggaattttcccattacctgctggagatggattgcttacacctgatgctcagaaaggaggagagacccctggatctgagcaatggaaatttcaggaattaagtcaaccacgttctcataccagcctgaaggatgagctttctgatgttagccaaggcggatctaaagctaccactccagcatcaacagctaattcagatgtggcaacaattcctactgatactcccttaaaggaagaaaacgaaggatttgtgaaggttacagatgcgccaaataaatcagagataagcaaacacattgaagtacaggtagcccaggaaactagaaatgtatctactggctctgctgaaaatgaagaaaagtcagaagttcaagcaatcatcgaatctactcctgagctggatatggacaaagatctcagtggatataaaggttcaagcactcccaccaaaggcatagagaacaaagcttttgatcgcaatacagaatctctctttgaagaactgtcttcagctggctcaggcctaataggagatgtggatgaaggagcagatttactaggaatgggtcgggaagttgagaatcttatattagaaaatacacaactgttggaaaccaaaaatgctttgaacatagtgaagaatgatttgatagcaaaagtggatgaactgacctgtgagaaagatgtgctgcaaggggaattggaggctgtgaagcaagccaaactgaaactagaggaaaagaacagagaattggaggaagagcttaggaaagctcgggcagaagctgaagatgcaaggcaaaaagcaaaagatgacgatgatagtgatattcccacagcccagaggaaacggtttactagagtagaaatggcccgtgttctcatggagcgaaaccagtataaagagagattgatggagcttcaggaagctgttcgatggacagagatgattcgggcatcacgagaaaatccagccatgcaggaaaaaaaaaggtcaagcatttggcagtttttcagccgacttttcagctcctcaagtaacacgactaagaagcctgaaccacctgttaatctgaagtacaatgcacccacgtctcatgttactccgtccgtcaagaaaagaagcagcaccttatctcagctccctggggataagtccaaagcctttgatttccttagtgaagaaactgaagctagtttagcctcacgcagagaacaaaagagagagcagtatcgtcaggtaaaagcacatgttcagaaggaagacggtagagtgcaggcttttggctggagtctgcctcagaagtacaaacaggtaaccaatggtcaaggtgaaaataagatgaaaaatttacctgtgcctgtctatctcagacctctggataaaaaagatacatcaatgaagctgtggtgtgctgttggagtcaatttatctggtgggaagaccagagatggtggttctgttgttggagcaagtgtattttacaaggatgttgctggtttggatacagaaggcagtaaacagcgaagtgcctctcagagtagtttagataagttagatcaggaacttaaggaacagcagaaggagttaaaaaatcaagaagaattatccagtctagtttggatctgtaccagcactcattcggctacaaaagttcttattattgatgctgttcaacctggcaacatcctagacagtttcactgtttgcaactctcatgttctgtgcattgcaagtgtgccaggtgcacgagaaacagactaccctgcaggagaagatctttcagaatctggtcaggtagacaaagcatctttatgtggaagtatgacaagcaacagctcagcagagacagacagcctgttaggaggcatcacagtggttggttgttctgcagaaggtgtgacgggagctgccacttcccctagtacaaatggtgcttctccagtgatggataaaccaccagaaatggaagcagaaaatagtgaggttgatgaaaatgttccaacagcagaagaagcaactgaagctacagaagggaatgcggggtcagctgaagacacagtggacatctcccaaactggcgtctacacagagcatgtctttacagatcctttgggagttcagatcccagaagacctctccccagtgtatcagtcgaggtataataatgggtcatcaacttaggcaactgggtgaagtgcacaaataaaaggaaccaaaataatctcaaacagtcaaaagatttccactaattagtgtcactgtggtggattaaaagatacctattcttgtgata

SEQ ID NO: 2 - protein, 766 aa, Homo sapiens
MIHNYMEHLERTKLHQLSGSDQLESTAHSRIRKERPISLGIFPLPAGDGLLTPDAQKGGETPGSEQWKFQELSQPRSHTSLKDELSDVSQGGSKATTPASTANSDVATIPTDTPLKEENEGFVKVTDAPNKSEISKHIEVQVAQETRNVSTGSAENEEKSEVQAIIESTPELDMDKDLSGYKGSSTPTKGIENKAFDRNTESLFEELSSAGSGLIGDVDEGADLLGMGREVENLILENTQLLETKNALNIVKNDLIAKVDELTCEKDVLQGELEAVKQAKLKLEEKNRELEEELRKARAEAEDARQKAKDDDDSDIPTAQRKRFTRVEMARVLMERNQYKERLMELQEAVRWTEMIRASRENPAMQEKKRSSIWQFFSRLFSSSSNTTKKPEPPVNLKYNAPTSHVTPSVKKRSSTLSQLPGDKSKAFDFLSEETEASLASRREQKREQYRQVKAHVQKEDGRVQAFGWSLPQKYKQVTNGQGENKMKNLPVPVYLRPLDKKDTSMKLWCAVGVNLSGGKTRDGGSVVGASVFYKDVAGLDTEGSKQRSASQSSLDKLDQELKEQQKELKNQEELSSLVWICTSTHSATKVLIIDAVQPGNILDSFTVCNSHVLCIASVPGARETDYPAGEDLSESGQVDKASLCGSMTSNSSAETDSLLGGITVVGCSAEGVTGAATSPSTNGASPVMDKPPEMEAENSEVDENVPTAEEATEATEGNAGSAEDTVDISQTGVYTEHVFTDPLGVQIPEDLSPVYQSRYNNGSST

(19) United States
(12) Patent Application Publication
(10) Pub. No.: US 2008/0220538 A1
(43) Pub. Date: Sep. 11, 2008

(54) Novel Cancer Associated Antibodies And Their Use In Cancer Diagnosis

(75) Inventor: Anil Kumar Suri, New Delhi (IN)

Correspondence Address: MERCHANT & GOULD PC, P.O. BOX 2903, MINNEAPOLIS, MN, 55402-0903, US

(73) Assignee: NATIONAL INSTITUTE OF IMMUNOLOGY, New Delhi (IN)

(21) Appl. No.: 11/664,881

(22) PCT Filed: Oct. 7, 2005
(86) PCT No.: PCT/IB05/02972

§ 371 Date: Nov. 7, 2007

(30) Foreign Application Priority Data
Oct. 7, 2004 (IN) 1945/DEL/2004

(51) Int. Cl. C07K 16/30; G01N 33/543; G01N 33/548; G01N 33/552

(52) U.S. Cl. 436/527; 530/387.9; 436/518; 436/530

(57) ABSTRACT

The present invention relates to an antibody having specificity for a polypeptide comprising an amino acid sequence of SEQ ID No:2 or an isoform thereof or a polypeptide comprising SEQ ID NO:2.

### FIELD OF THE INVENTION

The present invention relates to novel cancer associated antibodies and antigens and their use in cancer diagnosis.

### BACKGROUND OF THE INVENTION

Cancer, as is known is a killer disease and is a subject of investigation and research by several workers in various countries. It is observed that in most cases, cancer is diagnosed and treated only in the advanced stage, when the cancer cells have already invaded and metastasised throughout the body. More than 60% of patients with breast, lung, colon and ovarian cancer already have hidden or overt metastatic colonies. At this stage, therapeutic modalities are limited in their success. Detecting cancers in early stages even in the premalignant state, means that current or future treatment modalities might have a higher likelihood of a true cure.

Ovarian cancer is a prime example of this clinical dilemma. More than two-thirds of cases of ovarian cancer are detected at an advanced stage, when the ovarian cancer cells have spread away from the ovary and have disseminated throughout the peritoneal cavity [Gure, A. O. Altorki, N. K. Stockert E, Scanlan M. J., Old L. J, & Chen Y. T. (1998) Cancer Res 58, 1034-1341]. Although the disease at this stage is advanced, it rarely produces specific or diagnostic symptoms. Consequently, ovarian cancer is usually treated when it is at an advanced stage. The resulting five year survival rate is 35-40% for late-stage patients who receive the best possible surgical and chemotherapeutic intervention.

By contrast, if ovarian cancer is detected when it is still confined to the ovary (stage I), conventional therapy produces a high rate (95%) of five-year survival. Hence, early detection of ovarian cancer lacks a specific symptom, a specific biomarker and accurate and reliable diagnostic, non-invasive modalities.

Traditional methods of treating cancer include use of non-invasive or mildly invasive diagnostic tests, biopsy, histological examination and so on. However, each of these tests have their own limitations and hence the search for new methods of diagnosis and treatment of cancer.

In keeping with the above, it is desirable to provide an effective, clinically useful biomarker measurable in a readily accessible body fluid and tissues. Clinical proteomics is well suited to discovery of such biomarkers, as serum is a protein-rich information reservoir that contains the traces of what has been encountered by the blood during its constant perfusion and percolation throughout the body.

However, until now, the search for cancer-related biomarkers for early disease detection has been a ‘one-at-a-time’ approach, which has looked for over-expressed proteins in blood that are shed into the circulation as a consequence of the disease process [Adam, B. L., Vlahou, A., Semmes, O. J. & Wright, G. L. Jr. (2001) Proteomic approaches to biomarker discovery in prostate and bladder cancers. Proteomics 1, 1264-1270; Carter, D. et al. (2002) Purification and characterization of the mammaglobin/lipophilin B complex, a promising diagnostic marker for breast cancer, Biochemistry 41, 6714-6722; Rosty, C. et al (2002) Identification of hepatocarcinoma-intestinepancreas/pancreatitis-associated protein I as a biomarker for pancreatic ductal adenocarcinoma by protein biochip technology. Cancer Res. 62, 2868-1875; Xiao, Z. et al. (2001) Quantitation of serum prostate-specific membrane antigen by a novel protein biochip immunoassay discriminates benign from malignant prostate disease. Cancer Res. 61, 6029-6033; Kim, J. H. et al (2002) Osteopontin as a potential diagnostic biomarker for ovarian cancer. JAMA 287, 1671-1679]. Thus, the art so far has provided markers that are tissue specific and assist in identification of a specific type of cancer. None of them serve as universal markers or have universal application.

Hence, there is a need in art to develop diagnostic agents that may be used as markers having universal application and thus useful for detection of any type of cancer. To fulfil this need, the invention provides novel antibodies against SEQ ID NO:2, its isoform or a polypeptide comprising SEQ ID NO:2 and its use in diagnosis of cancer.

### OBJECTS OF THE INVENTION

The main object of the invention is to provide a novel diagnostic agent useful for detection of cancer. Another object is to provide a kit and methods for detection of cancer.

### DESCRIPTION OF THE INVENTION

The inventor during some of their investigations had found a novel gene encoding sperm associated antigen 9 (SPAG9) [labelled herein as SEQ ID NO: 1] which has been cloned from human testis cDNA library. The 2523 bp cDNA ([Shankar, Mohapatra and Suri (1998) Biochem. Biophys. Res. Commun. 243, 561-565; Acc. No: X91879] contains five direct repeats, three mirror repeats, three possible stem loop structures and three palindromic motifs. Open reading frame encodes a protein of 766 amino acids. The deduced protein analysis revealed several features: 1) a characteristic leucine zipper motif (LZ); 2) an extended coiled-coil domain (coil) and 3) a transmembrane domain (T). The amino acid sequence of SPAG9 (labelled herein as SEQ ID NO:2) further revealed that primary sequence identity to JNK binding domain. The secondary structural analysis revealed that SEQ ID NO:2 has an α-helical structure. CD spectra analysis further supported a predominant α-helical structure of SEQ ID NO:2. Microsequencing of oligomeric aggregates of recombinant SEQ ID NO:2 by tandem mass spectrometry confirmed the amino acid sequence and mono atomic mass to 83.9 kDa. The studies also revealed that SEQ ID NO:1 and 2 play a role in sperm-egg interaction. The said SEQ ID NO:1 was reported as a member of a new family of testis specific genes with a specific function and considered as a possible contraceptive target.

Thereafter, while the inventor was conducting certain unrelated studies on the MAPK (mitogen activated protein kinases pathway) and the involvement of proteins in this pathway, the inventor surprisingly found that SEQ ID NO:2 interacted with JNK signalling pathway. In fact, it had higher binding affinity to JNK3 and JNK2 compared with JNK1. No interaction was observed with p38α or ERK pathways. The said MAPK studies suggested that SEQ ID NO:2 molecule may be involved in cell-signalling pathway supporting cellular growth and cellular proliferation. This lead the inventor to investigate further the role of SEQ ID NO:2 in cancerous tissues as testis also involves a continuous proliferation of cells, almost mimicking the cancerous cell growth.

Surprisingly, the inventor found SEQ ID NO:1 nucleotide homology with various cancer tissue ESTs. In fact when tissue distribution of SEQ ID NO:1 was studied using different tissues, the inventors further found that mRNA of SEQ ID NO:1 is expressed exclusively in normal testis tissue. However, biological samples from various cancer patients revealed the presence of SEQ ID NO:2 in cancerous tissues such as lung, breast, stomach, uterus, esophagus, colon, ovarian, testis, cervix, skin, prostate, oral, bladder, endometrial, kidney, liver, brain, blood, vulva, vagina, gall bladder, eye and bone.

While not wishing to be bound by any theory, the inventor believes that SEQ ID NO:2 polypeptide peptide expressed in the aforesaid cancerous tissues may be recognized as a non-self protein by the immune system and may mount an auto-antibody response in such patients resulting in anti-SEQ ID NO:2 antibodies circulating in the blood stream of cancer patients. Such antibodies may provide a basis for early detection of cancer.

### Anti-SEQ ID NO:2 Antibodies

Accordingly, in one aspect, the invention provides a novel antibody or an antigen-binding fragment thereof, having specificity for a polypeptide bearing SEQ ID No:2, or an isoform thereof or a polypeptide comprising the said SEQ ID NO:2. The said antibody may be an IgG antibody or IgM antibody or a single chain or a fragment of the said antibody having specificity to the said polypeptide of SEQ ID NO:2.

Such antibodies serve as markers as they appear to be found in subjects suffering from cancer. The said antibodies of the invention are termed as “anti-SEQ ID No:2 antibody” and referred to as such hereafter. The anti-SEQ ID No:2 antibody as referred includes the antigen-binding fragment thereof. The term “antibody”, as used herein includes a polyclonal antibody, a monoclonal antibody, a humanized antibody and a single chain antibody.

The invention also provides a method for production of polyclonal anti-SEQ ID No:2 antibody comprising the steps of:
providing purified polypeptide of SEQ ID NO: 2 or synthesizing the same recombinantly by cloning and expressing SEQ ID NO:1 nucleic acid sequence in a suitable host;
mixing SEQ ID NO:2 with an adjuvant to obtain a composition,
injecting the composition into an animal to as produce an immune response, and
collecting the sera from the animal and isolating polyclonal antibody generated from the same.

In the method described above, the sera from the animal may be used directly or purified prior to use by various methods including affinity chromatography employing Protein A-Sepharose, antigen Sepharose or anti-mouse-Ig-Sepharose.

### Antibody Detection Kit

In another aspect, the invention provides a kit useful for detection of cancer in a biological sample of a subject suspected of or suffering from cancer. The said kit comprises a polypeptide sequence of SEQ ID NO:2 or an isoform thereof or a polypeptide comprising the said SEQ ID NO:2 as described herein above coupled to a solid matrix and instructional material. The solid matrix as referred herein may include nitrocellulose paper, glass slide, microtitre plates and wells.

In yet another aspect, the invention provides method for detecting a cancer in a subject suffering from or suspected of suffering from a cancer, comprising the steps of:
providing a biological sample of a subject suspected of cancer; and
detecting the presence of antibodies generated against SEQ ID NO:2 or an isoform thereof or a polypeptide comprising SEQ ID NO:2 in the sample,
   wherein presence of antibodies generated against SEQ ID NO:2 or an isoform thereof or a polypeptide comprising the said SEQ ID NO:2 is indicative of cancer.

In another aspect, the invention provides method for detecting a cancer in a subject suffering from or suspected of suffering from a cancer, comprising the steps of:
providing a biological sample of a subject suspected of cancer;
contacting the sample with the kit as set forth above and determining binding of SEQ ID NO:2 or an isoform thereof or a polypeptide comprising the said SEQ ID NO:2 with anti-SEQ ID No:2 antibody if present in biological sample through a detectable signal, which is indicative of cancer.

The detectable signal as referred herein may be any detectable signal such as development of color or fluorescence. For example, a secondary labelled antibody (enzyme conjugated antibody) may be introduced which may bind to anti-SEQ ID No:2 antibody if present in patient's sample and provide a detectable signal which may be used as a parameter for detection of cancer.

### Antigen-Detection Kit

The antigen detection kit comprises an antibody having specificity to a polypeptide sequence bearing SEQ ID NO:2 or an isoform thereof or a polypeptide comprising SEQ ID NO:2. The kit is useful for detection of cancer.

In yet another aspect, the invention provides method for detecting a cancer in a subject suffering from or suspected of suffering from a cancer, comprising the steps of:
providing a biological tissue of a subject suspected of cancer; and
detecting the presence of sequence of SEQ ID NO:2 polypeptide or an isoform thereof or a polypeptide comprising SEQ ID NO:2 in the tissue,
   the presence of which is indicative of cancer.

In another aspect, the invention provides method for detecting a cancer in a subject suffering from or suspected of suffering from a cancer, comprising the steps of:
providing a biological tissue of a subject suspected of cancer;
contacting the tissue with the antigen-detecting kit as set forth above and determining binding of polypeptide sequence bearing SEQ ID NO:2 [or an isoform thereof or a polypeptide comprising SEQ ID NO:2] with anti-SEQ ID No:2 antibody through a detectable signal, which is indicative of cancer.

The detectable signal as referred herein may be any detectable signal such as development of color or fluorescence. For example, a secondary labelled antibody (enzyme conjugated antibody) may be introduced which may bind to the to the anti-SEQ ID No:2 antibody if present in patient's sample and provide a detectable signal which may be used as a parameter for detection of cancer. Yet another example is where the anti-SEQ ID No:2 antibody employed in the said kit may itself be a labelled antibody (labelled for color or fluorescence) and detectable signal produced may be used as a parameter for detection of cancer.

The term ‘biological sample’ as used herein refers to fluids or tissues obtained from subjects suspected of or suffering from cancer. Examples include blood, serum, plasma, tissue sample, urine and saliva. Further, ‘cancer’ as used herein refers to cancer of ectodermal, endodermal or mesodermal origin. The said cancer may be any cancer such as that of lung, breast, stomach, uterus, esophagus, colon, ovarian, testis, cervix, skin, prostate, oral, bladder, endometrial, kidney, liver, brain, blood, vulva, vagina, gall bladder, eye and bone. The ‘subject’ as referred above includes any mammal.

A few embodiments of the invention are now illustrated by the following examples, and drawings:

### BRIEF DESCRIPTION OF THE ACCOMPANYING DRAWINGS

FIG. 1 is a Dot Blot analysis comparing a control with patient's samples.

FIG. 2 which is a Western Blot analysis comparing a control with patient's samples.

FIG. 3 is the ELISA assay which indicates antibody titres in cancer patient's and healthy biological samples.

FIG. 4 represents the immunohistochemical analysis comparing the tissues obtained from normal healthy control and different cancer patients.

The examples below are meant for illustration only and are not intended as limitations on the inventive scope thereof. Various adaptations and variations that are possible in keeping with the inventive concept of the invention are deemed to fall within the scope of the invention.

### EXAMPLE 1

### Antibody Detection Kit

### Preparation of Recombinant SEQ ID NO:2 Protein

The applicant conducted several studies based on the findings of the invention and have now identified the SEQ ID NO:2 polypeptide and which cloned in a suitable vector for large scale expression and purification as under:

### Antigen Preparation: Plasmid Construction

Recombinant SEQ ID NO:2 protein (complete open reading frame encoding 766 amino acids) was successfully cloned in pET 28 b(+) expression vector. Briefly, a cDNA encoding a complete open reading frame of SEQ ID NO:1 was amplified using suitable primers. The product was digested with restriction enzymes and was inserted into digested pET28b(+) vector (Novagen, Madison, USA) containing multiple cloning sites to obtain pET28b− SEQ ID NO:2.

Expression of SEQ ID NO:2 Protein in Escherichia coli

Competent cells were prepared and transformed with pET28b− SEQ ID NO:2. Transformed cells were used to inoculate LB media for growing E. coli culture. Expression of recombinant SEQ ID NO:2 was induced with IPTG (isopropyl-β-D-thiogalactopyranoside) and subsequently purified.

Purification of SEQ ID NO:2 Expressed in Escherichia coli

E. coli culture expressing SEQ ID NO:2 was harvested, washed and sonicated using sonication buffer containing urea. The sonicated sup was loaded on to the agarose beads column charged with nickel sulphate. The column was washed with washing buffers and elutes were collected using elution buffer. The purified SEQ ID NO:2 recombinant protein analysed for amino acid sequence by Mass Spectrometry. Amino acid sequencing analysis revealed that the recombinant SEQ ID NO:2 protein was expressed as deduced amino acid sequence published (EMBL Acc. No. 91879, Shankar et al., 1998).

### Antibody-Detection Kit:

An antibody detection kit comprising SEQ ID NO:2 bound to a solid matrix is prepared as under:

A drop of purified SEQ ID NO:2 protein was placed on to the nitrocellulose membrane, fixed with methanol and air dried. The dipstick was dipped into transfer buffer and then air dried again. Dot blot analysis was performed by using dipsticks, which are made of polyester backing material with a nitrocellulose membrane.

To determine anti-SEQ ID No:2 antibody in the various biological samples from different cancer patients, the strip coated with SEQ ID NO:2 was dipped into the patient's biological fluid such as serum, followed by anti-human HRPO as secondary antibody (which was obtained from a commercial source). The dipstick was taken out and washed with PBS buffer. Finally, the strip was treated with 0.05% 3,3′-Diaminobenzidene (DAB) (Sigma).

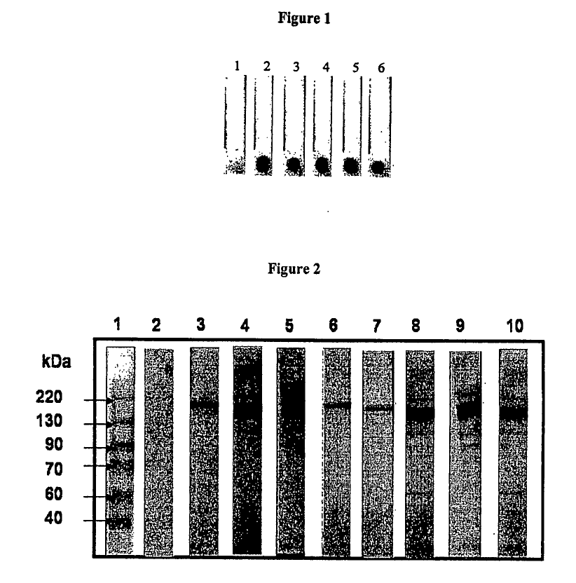

In the dot blot analysis, the appearance of a dark brown reaction on nitrocellulose membrane indicated presence of anti-SEQ ID No:2 antibody in the sample. As compared to the samples obtained from cancer patients, the samples obtained from normal human subjects did not show any reaction. The results are shown in FIG. 1.

As shown in FIG. 1, biological samples from cancer patients exhibit strong reaction as compared to a healthy subject. Lane 1 represents the reaction with serum from normal subject—there is no development of any color. Lane 2 represents reaction with serum of cervix cancer, Lane 3 indicates reactivity of bladder cancer patient's serum, Lane 4 depicts the reaction of serum obtained from colon cancer patient, Lane 5 demonstrates the reaction of breast cancer serum, and Lane 6 is the reactivity of serum from ovarian cancer. Development of dark brown colour as in lanes 2 to 6 is indicative of cancer.

### Gel Electrophoresis and Western Blotting

The presence of anti-SEQ ID No:2 antibodies-in a biological sample may also be detected by western blotting procedure wherein recombinant protein is run on SDS PAGE and transferred onto nitrocellulose matrix:

Recombinant SEQ ID NO:2 protein was run on SDS polyacrylamide gel. Briefly, the protein solution was diluted with sample buffer. The samples were then loaded onto polyacrylamide gel. After electrophoresis, proteins were transferred onto nitrocellulose membrane. Blocked membrane was probed with cancer patients' serum and subsequently with anti-human HRPO (which was obtained from a commercial source) as secondary antibody. Finally, strip was treated with 0.05% DAB.

Western blot analysis of sample of a patient suffering from cancer demonstrated a strong reactivity between cancerous sample and recombinant SEQ ID NO:2 protein. A strong band of recombinant SEQ ID NO:2 was observed on the nitrocellulose membrane strips treated with cancer patients' sample, whereas sample from normal human subjects failed to show any reactivity. The results are depicted in FIG. 2 which is a western blot analysis comparing a control with patient's samples. As shown in FIG. 2, lane 1 represents molecular weight marker, lane 2 represents sample obtained from normal person, lane 3 is the sample obtained from a subject with oral cancer, Lane 4—bladder cancer, lane 5 lung cancer, lane 6 prostrate cancer, lane 7 uterus, lane 8 ovary, lane 9 colon cancer and lane 10 breast cancer. As may be seen in the Western blot analysis, lanes 3 to 10 show presence of a band of 170 kDa which is absent in lane 2 of normal person.

### Enzyme Linked Immunosorbent Assay (ELISA)

Further, the presence of anti-SPAG9 antibodies in a biological sample may also be detected by ELISA Technique as under:

Microtitration plates (Nunc, Rosaklide, Denmark) were coated with recombinant SEQ ID NO:2 protein for ELISA assay. Post blocking, the plates were incubated with the samples from cancer patients' and/or healthy control. Bound antibodies were revealed with anti-human immunoglobulins conjugated to horseradish peroxidase (which was obtained from a commercial source). Enzyme activation was carried out with 0.05% orthophenylenediamine as the substrate. The antibody response of cancer patients and healthy control individuals was represented as the mean of the absorbance of the sample of the individuals.

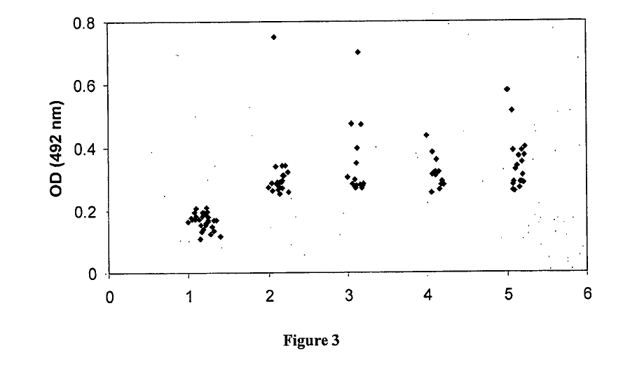

FIG. 3 is the ELISA assay which indicates antibody titres in cancer patient's and healthy biological samples. The absorbance values are indicated on Y-axis whereas different cancer types are depicted as numerals on the X-axis. (1) represents the titres obtained from serum samples from normal subject, (2) represents the titres obtained from cervix cancer patients, (3) denotes the titre values from patients suffering from bladder cancer, (4) gastrointestinal tract cancer, (5) ovarian cancer. The test sample titre values are considered indicative of cancer when the estimated ELISA titres are above the mean +2SD of healthy biological sample. Cancer patient's biological sample revealed higher ELISA titre values above the mean +2SD of healthy sample as indicated in the figure, which is indicative of cancer (+2SD is the standard cut off).

TABLE 1
Samples tested & Results:

| | Dot blot | | Western blot | | | |
|---|---|---|---|---|---|---|
| | analysis | | analysis | | ELISA | |
| Biological | | can- | | can- | | can- |
| sample | Normal | cerous | Normal | cerous | Normal | cerous |
| Lung | − | + | − | + | − | + |
| Breast | − | + | − | + | − | + |
| Prostrate | − | + | − | + | − | + |
| Bladder | − | + | − | + | − | + |
| Utreus | − | + | − | + | − | + |
| Esophagus | − | + | − | + | − | + |
| Testis | − | + | − | + | − | + |
| Vulva | − | + | − | + | − | + |
| Liver | − | + | − | + | − | + |
| Eye | − | + | − | + | − | + |
| Oral | − | + | − | + | − | + |
| Kidney | − | + | − | + | − | + |
| Colon | − | + | − | + | − | + |
| Cervix | − | + | − | + | − | + |
| Vagina | − | + | − | + | − | + |
| Brain | − | + | − | + | − | + |
| Blood | − | + | − | + | − | + |
| Bone | − | + | − | + | − | + |
| Stomach | − | + | − | + | − | + |
| Ovarian | − | + | − | + | − | + |
| Skin | − | + | − | + | − | + |
| Gall bladder | − | + | − | + | − | + |

### EXAMPLE 2

### Antigen Detection Kit

Preparation of Anti-SEQ ID No:2 Antibodies: Immunization of Rats with Recombinant SEQ ID NO:2 protein

For generating anti-SEQ ID NO:2 antibodies, adult female rats were employed. Rats were immunized with SEQ ID NO:2 protein with a primary and two booster injections at different time intervals. First immunization of was done with SEQ ID NO:2 mixed with an adjuvant. Following booster injections were carried out at weekly intervals, rats are then bled and the sera isolated.

Purification of Anti-SEQ ID No:2 Antibodies from Rat Sera

The sera can be used directly or purified prior to use by various methods including affinity chromatography employing protein beads. Column containing protein A beads was loaded with rat sera and kept on the shaker for overnight. The column was centrifuged and flow through was collected. Subsequently, column was washed with binding buffer and finally elutes were collected after centrifugation. The concentration of antibodies was calculated by taking OD at 280 nm.

Thus the antigen detection kit was prepared comprising anti-SEQ ID No:2 antibodies, for detection of SEQ ID NO:2 protein in tissues: The details of the immuno-histochemistry analysis performed is as below:

Tissues from cancerous patients were fixed by standard fixative using standard Immunohistochemistry procedure. Biological tissue sections were incubated with rat anti-SEQ ID No:2 antibody and then treated with goat anti-rat immunoglobulins conjugated to horseradish peroxidases (which was obtained from a commercial source). After washing, sections were treated with 0.01% DAB and were counter-stained with Mayer's hematoxylin.

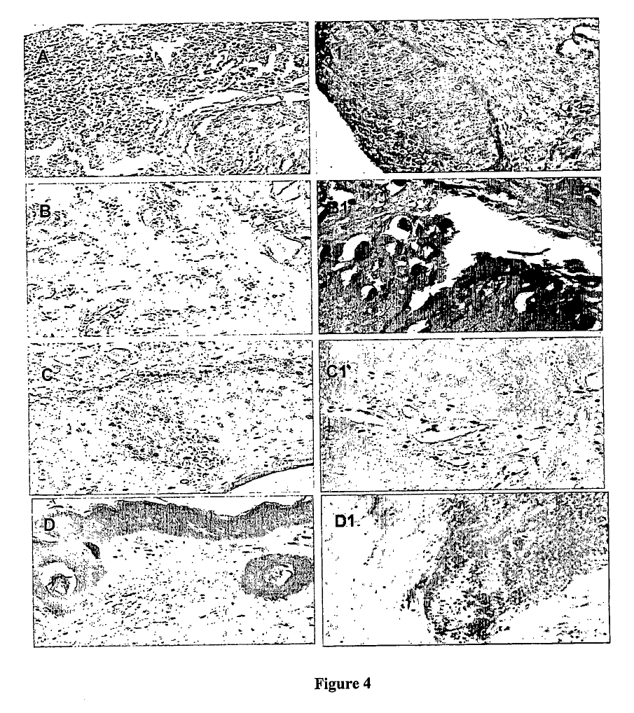

FIG. 4 represents the immunohistochemical analysis comparing the tissues obtained from normal healthy control and different cancer patients. The normal tissues are in the left lane while the cancerous tissues are depicted on right side.

(A) is the immunohistochemical analysis of cervix tissue obtained from normal subject, (A1) is the tissue obtained from subject suffering form cervix cancer; (B) is immunohistochemical analysis of normal mesenchymal tissue and (B1) is the tissue obtained from the subject suffering from mesenchymal cancer; (C) is the normal tongue tissue and (C1) is the tongue cancerous tissue; (D) is the normal skin tissue and (D1) is the skin cancerous tissue;

A strong reactivity between SEQ ID NO:2 and the anti-SEQ ID No:2 antibody was observed in cancer tissues whereas tissues from normal healthy controls did not reveal any reactivity. The development of brown color in the cancer tissues is indicative of cancer in the given subject.

### Samples Tested:

Several cancerous tissues were examined for the expression of SEQ ID NO:2 protein and the results are set out below:

Antigen detection Kit

| Tissue type | Normal | cancerous |
|---|---|---|
| Lung | − | + |
| Breast | − | + |
| Prostrate | − | + |
| Bladder | − | + |
| Utreus | − | + |
| Esophagus | − | + |
| Testis | + | + |
| Vulva | − | + |
| Liver | − | + |
| Eye | − | + |
| Oral | − | + |
| Kidney | − | + |
| Colon | − | + |
| Cervix | − | + |
| Vagina | − | + |
| Brain | − | + |
| Blood | − | + |
| Bone | − | + |
| Stomach | − | + |
| Ovarian | − | + |
| Skin | − | + |
| Gall bladder | − | + |

The strong reactivity of anti-SEQ ID No:2 antibodies with the cancerous tissues demonstrated the expression of SEQ ID NO:2 in various cancer cells. Since SEQ ID NO:2 is exclusively expressed in the normal testis, the presence of SEQ ID NO:2 in the tissues other than testis accounts for the onset/development of malignancy in the tissues.

| SEQ ID NO:1 Nucleotide sequence | |
|---|---|
| 1 | gacaacttga gctgaaagcg aaaaactatg ctgaccagat tagcagactt gaagaaagag |
| 61 | aagcagaact gaagaaggaa tataatgcat tacatcaaag acacactgag atgatccata |
| 121 | attatatgga acatttagaa agaacaaaac ttcatcagct ctcagggagt gatcaactag |
| 181 | aatccacagc tcatagtaga attagaaaag aacgccctat atcattagga attttcccat |
| 241 | tacctgctgg agatggattg cttacacctg atgctcagaa aggaggagag acccctggat |
| 301 | ctgagcaatg gaaatttcag gaattaagtc aaccacgttc tcataccagc ctgaaggatg |
| 361 | agctttctga tgttagccaa ggcggatcta aagctaccac tccagcatca acagctaatt |
| 421 | cagatgtggc aacaattcct actgatactc ccttaaagga agaaaacgaa ggatttgtga |
| 481 | aggttacaga tgcgccaaat aaatcagaga taagcaaaca cattgaagta caggtagccc |
| 541 | aggaaactag aaatgtatct actggctctg ctgaaaatga agaaaagtca gaagttcaag |
| 601 | caatcatcga atctactcct gagctggata tggacaaaga tctcagtgga tataaaggtt |
| 661 | caagcactcc caccaaaggc atagagaaca aagcttttga tcgcaataca gaatctctct |
| 721 | ttgaagaact gtcttcagct ggctcaggcc taataggaga tgtggatgaa ggagcagatt |
| 781 | tactaggaat gggtcgggaa gttgagaatc ttatattaga aaatacacaa ctgttggaaa |
| 841 | ccaaaaatgc tttgaacata gtgaagaatg atttgatagc aaaagtggat gaactgacct |
| 901 | gtgagaaaga tgtgetgcaa ggggaattgg aggctgtgaa gcaagccaaa ctgaaactag |
| 961 | aggaaaagaa cagagaattg gaggaagagc ttaggaaagc tcgggcagaa gctgaagatg |
| 1021 | caaggcaaaa agcaaaagat gacgatgata gtgatattcc cacagcccag aggaaacggt |
| 1081 | ttactagagt agaaatggcc cgtgttctca tggagcgaaa ccagtataaa gagagattga |
| 1141 | tggagcttca ggaagctgtt cgatggacag agatgattcg ggcatcacga gaaaatccag |
| 1201 | ccatgcagga aaaaaaaagg tcaagcattt ggcagttttt cagccgactt ttcagctcct |
| 1261 | caagtaacac gactaagaag cctgaaccac ctgttaatct gaagtacaat gcacccacgt |
| 1321 | ctcatgttac tccgtccgtc aagaaaagaa gcagcacctt atctcagctc cctggggata |
| 1381 | agtccaaagc ctttgatttc cttagtgaag aaactgaagc tagtttagcc tcacgcagag |
| 1441 | aacaaaagag agagcagtat cgtcaggtaa aagcacatgt tcagaaggaa gacggtagag |
| 1501 | tgcaggcttt tggctggagt ctgcctcaga agtacaaaca ggtaaccaat ggtcaaggtg |
| 1561 | aaaataagat gaaaaattta cctgtgcctg tctatctcag acctctggat aaaaaagata |
| 1621 | catcaatgaa gctgtggtgt gctgttggag tcaatttatc tggtgggaag accagagatg |
| 1681 | gtggttctgt tgttggagca agtgtatttt acaaggatgt tgctggtttg gatacagaag |
| 1741 | gcagtaaaca gcgaagtgcc tctcagagta gtttagataa gttagatcag gaacttaagg |
| 1801 | aacagcagaa ggagttaaaa aatcaagaag aattatccag tctagtttgg atctgtacca |
| 1861 | gcactcattc ggctacaaaa gttcttatta ttgatgctgt tcaacctggc aacatcctag |
| 1921 | acagtttcac tgtttgcaac totcatgttc tgtgcattgc aagtgtgcca ggtgcacgag |
| 1981 | aaacagacta ccctgcagga gaagatcttt cagaatctgg tcaggtagac aaagcatctt |
| 2041 | tatgtggaag tatgacaagc aacagctcag cagagacaga cagcctgtta ggaggcatca |
| 2101 | cagtggttgg ttgttctgca gaaggtgtga cgggagctgc cacttcccct agtacaaatg |
| 2161 | gtgcttctcc agtgatggat aaaccaccag aaatggaagc agaaaatagt gaggttgatg |
| 2221 | aaaatgttcc aacagcagaa gaagcaactg aagctacaga agggaatgcg gggtcagctg |
| 2281 | aagacacagt ggacatctcc caaactggcg tctacacaga gcatgtcttt acagatcctt |
| 2341 | tgggagttca gatcccagaa gacctctccc cagtgtatca gtcgaggtat aataatgggt |
| 2401 | catcaactta ggcaactggg tgaagtgcac aaataaaagg aaccaaaata atctcaaaca |
| 2461 | gtcaaaagat ttccactaat tagtgtcact gtggtggatt aaaagatacc tattcttgtg |
| 2521 | ata |
| SEQ ID NO:2 AMINO ACID SEQUNCE | |
| 1 | mihnymehle rtklhqlsgs dqlestahsr irkerpislg ifplpagdgl ltpdaqkgge |
| 61 | tpgseqwkfq elsqprshts lkdelsdvsq ggskattpas tansdvatip tdtpLkeene |
| 121 | gfvkvtdapn kseiskhiev qvaqetrnvs tgsaeneeks evqaiiestp eldmdkdlsg |
| 181 | ykgsstptkg ienkafdrnt eslfeelssa gsgligdvde gadllgmgre venhilentq |
| 241 | lletknalni vkndliakvd eltcekdvlq geleavkqak Ikleeknrel eeelrkarae |
| 301 | aedarqkakd dddsdiptaq rkrftrvema rvlmernqyk erlmelqeav rwtemirasr |
| 361 | enpamqekkr ssiwqffsrl fssssnttkk peppvnlkyn aptshvtpsv kkrsstlsql |
| 421 | pgdkskafdf lseeteasla srreqkreqy rqvkahvqke dgrvqafgws Ipqkykqvtn |
| 481 | gqgenkmknl pvpvylrpld kkdtsmklwc avgvnlsggk trdggsvvga svfykdvagl |
| 541 | dtegskqrsa sqssldkldq elkeqqkelk nqeelsslvw ictsthsatk vliidavqpg |
| 601 | nildsftvcn shvlciasvp garetdypag edlsesgqvd kaslcgsmts nssaetdsll |
| 661 | ggitvvgcsa egvtgaatsp stngaspvmd kppemeaens evdenvptae eateategna |
| 721 | gsaedtvdis qtgvytehvf tdplgvqipe dlspvyqsry nngsst |

1. An antibody having specificity for a polypeptide comprising an amino acid sequence of SEQ ID No:2 or an isoform thereof or a polypeptide comprising SEQ ID NO:2.

2. An antibody as claimed in claim 1 wherein the antibody is a monoclonal antibody, polyclonal antibody, humanised or recombinant antibody.

3. An antibody fragment having specificity for a polypeptide consisting of SEQ ID No:2 or an isoform thereof or a polypeptide comprising SEQ ID NO:2.

4. A single chain of the antibody fragment of claim 3.

5. An antibody as claimed in claim 1 wherein the antibody is an IgG antibody or IgM antibody.

6. A kit for detection of cancer in a biological sample of a subject suspected of or suffering from cancer, comprising polypeptide sequence bearing SEQ ID NO:2 or an isoform thereof or a polypeptide comprising SEQ ID NO:2 optionally coupled to a solid matrix and instructional material.

7. A kit as claimed in claimed in claim 6 wherein the solid matrix comprises nitrocellulose paper, glass slide, microtitre plates and wells.

8. A kit for detection of cancer in a biological sample of a subject suspected of or suffering from cancer comprising an antibody or a fragment thereof as claimed in claim 1.

9. A kit as claimed in claimed in claim 8 wherein the antibody is a labelled antibody.

10. A method for detecting a cancer in a subject suffering from or suspected of suffering from a cancer, comprising the steps of:
providing a biological sample of a subject suspected of cancer; and detecting the presence of an antibody generated against SEQ ID NO:2 or an isoform thereof or a polypeptide comprising SEQ ID NO:2, the presence of said antibodies being indicative of cancer.

11. A method for detecting a cancer in a subject suffering from or suspected of suffering from a cancer, comprising the steps of:
providing a biological sample of a subject suspected of cancer; and detecting the presence of polypeptide of SEQ ID NO:2 or an isoform or a polypeptide sequence comprising SEQ ID NO:2 therein.

12. A method for detecting a cancer in a subject suffering from or suspected of suffering from a cancer, comprising the steps of:
providing a biological sample of a subject suspected of cancer;
contacting the sample with a kit as claimed in claim 6
determining binding of SEQ ID NO:2 or an isoform thereof or a polypeptide sequence comprising SEQ ID NO:2 with antibody through a detectable signal which is indicative of cancer.

13. A method for detecting a cancer in a subject suffering from or suspected of suffering from a cancer, comprising the steps of:
providing a biological sample of a subject suspected of cancer;
contacting the sample with a kit as claimed in claim 8 and
determining the binding of SEQ ID NO:2 or an isoform thereof or a polypeptide sequence comprising SEQ ID NO:2 with antibody generated against the said polypeptide through a detectable signal which is indicative of cancer.

14. A kit as claimed in claim 6 wherein the cancer is of ectodermal, endodermal or mesodermal origin.

15. A kit as claimed in claim 6 wherein the cancer is of lung, breast stomach, esophagus, colon, ovarian, testis, cervix, skin, prostate, oral, bladder, liver, endometrial, kidney, brain, blood, vulva, vagina, gall bladder, eye and bone cancer.

16. A method as claimed in claim 10 wherein the subject is a mammal.

17. A method as claimed in claim 6 wherein the cancer is of ectodermal endodermal or mesodermal origin.
